(19) United States
(12) Patent Application Publication
(10) Pub. No.: US 2004/0116890 A1
(43) Pub. Date: Jun. 17, 2004

(54) Mechanical fastening system for absorbing articles

(76) Inventors: Francisco J. V. Hernandez, San Jose Dos Campos (BR); Carlos da S. Macedo Jr., Sao Jose Dos Campos (BR); Eduardo C.A. Aledo, Sao Jose Dos Campos (BR)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/471,933

(22) PCT Filed: Oct. 18, 2001
(86) PCT No.: PCT/BR01/00133

(30) Foreign Application Priority Data
Mar. 16, 2001 (BR) PI 0100992-3

(51) Int. Cl. A61F 13/15

(52) U.S. Cl. 604/387

(57) ABSTRACT

We describe herein a mechanical fastening system 5 for absorbing articles, such as disposable diapers (10) with an external layer or impermeable coating (12), an internal layer or permeable sheet (14) and an absorbing core of multiple layers sandwiched between the external coating (12) and the internal sheet (14), defining a frontal part (18) and a rear part (20) and a part between the legs (22) between the frontal and rear parts, the part between the parts with elastics (23) to adjust on the user's thigh region.

In the rear part (20) of the waist region (24) of 15 the absorbing article, between the external (12) and internal (14) layers, the elastic elements are added and adhered to and, with the liberation, a contraction occurs, forming a continuous region of corrugated filaments or loop fibers (28), adequate for the fastening of a plurality of hooks (32) disposed on the frontal part (18) of the absorbing article (10).

This invention is a mechanical fastening system 5 for absorbing articles. More specifically, this invention refers to absorbent items, such as disposable diapers, panties for training children, geriatric panties, panties for urinary incontinence, sanitary napkins and similar articles with enhanced systems.

### SCOPE OF THE INVENTION

Absorbing articles have an anatomic format suitable for the users in question, 80 that they may be adjusted to the body in order to receive and hold body liquids for a considerable period of use, being constituted by an impermeable layer on the external side, one layer permeable to liquid on the internal side in contact with the users, and a highly absorbable core with multiple layers, especially, in the region of discharge of body fluids, despite its reduced thickness.

### BACKGROUND OF THE PREVIOUS TECHNIQUE

Closing or fastening systems for absorbing articles are known in this technique, which uses a system of adhesive strips placed on the side edges of the article or, alternatively, Velcro or similar kinds of strips.

Fastening elements, such as adhesive strips or Velcro strips, include a pair of flaps that can be disposed both on the front part and on the rear of the absorbing article, and during its use, are superposed for closing. When, for example, the string is located on the rear of the absorbing article, it will be superposed on the front of it and vice versa.

Conventional systems of mechanical fastening are made of a pair of Velcro strips or strings, comprising a first strip with various hook elements and a second strip with various continuous elements, with interstitial empty spaces between such continuous elements, which form loops, suitable for fastening the various hooks in the first strip. The first and the second strips are fixed in opposite sides in the body or structure of the absorbing article, for example, disposable diaper.

A sheet of cloth or non-woven fabric of heavy 15 thickness was also developed in this technique, to be used as a strip of loop to facilitate the twisting with the strip of the hook. However, the manufacture of this sheet is extremely expensive, which makes its use unfeasible in the manufacture of the body of the absorbing article.

This fastening system with Velcro is also extremely expensive, besides requiring more precision at the placement of the fastening strings, since the fastening is only possible in a predetermined place, where the strips are attached to the body of the absorbing article. The correct closing and/or opening of the absorbing article at the moment of its placement or removal is rather difficult due to the restriction of the fastening area.

Although a reasonable fastening can be obtained with the previous technique, it was observed that they do not prevent the occurrence of leakage or displacement of the diaper, when the user moves especially, children, which leads the absorbing article to run from waist downwards, causing an unpleasant sensation to the user.

In an effort to prevent such running of the absorbing article, which tends to occur mainly on the front part, due to the weight of the absorbing article after the discharge of body fluids, additional elastic bands were used around the waist in order to achieve a better adjustment, which, in fact, allowed a better adjustment in this area, but did not prevent the fall of the article downwards after discharge of body fluids, since there is a considerable force vector downwards.

Another problem concerning the absorbing articles of the previous technique is that observed when the user moves himself or herself. Due to the place of Velcro fastening elements of the previous technique, the confluence of pelvic-joint bone exerts a rotation movement as the user moves. Accordingly, it causes the Velcro elements of fastening to get loose, an unacceptable problem for this kind of product.

An example of technique fastening system is that 25 described in U.S. Pat. No. 5,899,896, in which the fastening is achieved by a pair of Velcro strips disposed on the sides of the diapers, and a strip with hooks is placed in the internal part of the front cover and a string with loops is placed outside the front cover. This patent aim at alleviating the tension mentioned above with regard to the user's movement, with the use of the system that doses by superposition of the front part of the article backwards.

However, such system does not solve the problems observed with regard to the difficulties of placing the absorbing article due to the fixed region for the fastening of the absorbing article.

### BACKGROUND OF THE INVENTION

In this invention, the internal coating or sheet has a soft surface and does not irritate the skin of the user, it's hydrophilic, sufficiently porous to be permeable to liquid, allowing the liquid to penetrate without obstacles. A recommended coating may be produced among a huge quantity of fabrics, such as porous foams, reticulated foams, perforated films, natural fibers (such as wood or wool fibers), synthetic fibers (such as polyester fibers or polypropylene), or a combination of natural and synthetic fibers. The internal coating or sheet used in the absorbing article of this invention is suitable to isolate the user's skin with respect to liquids absorbed by the absorbing material in the core, avoiding the contact with the skin in the occurrence of discharge of body liquids.

Several fabric and non-woven may be used in the internal sheet, such as blown or carded polyolefin fibers, and natural and synthetic fibers.

The internal coating or sheet may be constituted of an essentially hydrophilic material, which can be treated with a surface-active substance, or otherwise, in order to reach the desirable level of absorption. In a particular test of this invention, the coating is of non-woven of polypropylene constituted of about 2.8 to 3.2 of denier fibers formed in a screen with a basic weight of about 22 grams per square meter and a density of about 0.06 grams per cubic centimeter. The fabric is treated on a surface of about 0.20 to 0.80 percent of the weight of a surface-active substance.

The external sheet is, preferably, of a material substantially impermeable to liquids, for example, a typical external sheet may be produced by a fine flexible film, or of another impermeable material impermeable to liquid. The external sheet prevents the humidity of the fluids in the absorbing material of the absorbing article core from soaking into the user's clothes. An example of external sheet is the polypropylene film with thickness of about 0.012 mm (0.5 mil) to 0.051 mm (2.0 mil).

### SUMMARY OF THE INVENTION

The problems noticed in the conventional closing systems led to the development of the closing system of this invent ion.

One of the purposes of this invention is to solve the problem of fastening at a predetermined place, in opposite parts to the external rim of the absorbing article around the waist, forming on each side of the referred article areas of predetermined fastening, herein called areas dedicated to closing. Such provision solves the limitation of placing for fixing the absorbing article, which besides being expensive, makes the use of such article difficult and prevents a correct adjustment to the region of the waist.

Another purpose of this invention is to solve the problem identified with respect to the rotation movement of the absorbing article due to the user's movement. In use, the predetermined areas of fastening are located on each side of the article, being disposed towards and imaginary central plan passing between the user's legs, causing the rotation of the article, and as a result, the hook and loop fastening system opens since they do not support the movement.

One of the purposes of the invention to solve the identified problems in the preceding technique is the provision of a fastening system for disposable articles having, around the waist, between the impermeable external sheet and the absorbing internal sheet, elastic materials that, whenever contracted, form a continues area of fibers corrugated and concentrated in the internal coating or sheet, which is more flexible than the impermeable cover, which provides a greater quantity of filaments or fibers for twisting or loop elements. The elastic material is tight between the tensioned internal and external sheets, with the purpose to keep the corrugation when it is released. The level of corrugation will depend on the stretching to which the elastic material is submitted, and the use of amounts between 1% and 100% of corrugation level will be possible.

This invention is intended to a mechanical fastening system that enables an easy, safe, reliable and stable fastening, guaranteeing comfort and protection of the absorbing article against undesirable leakage or detachment.

This invention has as an advantage the fact that the portion of the loop is an integral part of the waist area of structure, which provides for a limited adjustment to the user's body, with a reduced cost of production, since it's not necessary a separate manufacture of loop part which shall be tied on the surface of the structure of the absorbing article. The twisting power (force and energy) is substantially increased when the corrugation in the surface of the sheet is produced, as depicted in the graphs of FIGS. 6 to 11. Besides, the fastening system of this invention provides more reliability, once there is not the risk of the element or part of loop detaching from the structure of the absorbing article, which would make it useless. Also The fabric or non-woven sheet of high or low weight with introduction of elastic materials and the proposed corrugation, enables a tight grip of the same on the hook portion, which enables the absorbing article to get fixed to the user's body, preventing undesirable leakage and loosening.

The objectives of this invention are reached by a mechanical fastening system, especially for use on disposable diapers with a structure, comprising a hook portion with at least one twisting element and one loop portion with at least a twisting element, the hook portion and the loop portion twist on each other, and the loop portion being constituted by a sheet of fabric or non-woven substantially corrugated with the introduction of elastic materials.

### BRIEF DESCRIPTION OF THE DRAWINGS

This invention, described in full details below, 10 is based on a preferred modality represented in the attached drawings, among which:

FIG. 1 is a view in perspective of a disposable diaper, in position of use, open for best visualization, which is provided with a mechanical fastening system object of this invention;

FIG. 2 is a detailed schematic view of the hook elements of mechanical fastening subject of this invention;

FIG. 3 is a detailed schematic view of the loop elements of the mechanical fastening system provided in the part of the sheet of disposable diaper;

FIG. 4 is a view in perspective of the disposable diaper o FIG. 1, in position of use, closed;

FIG. 5 is a detailed schematic view of the loop elements and hook of the fastening system of this invention in position of use;

FIG. 6 is a graphic of the corrugation percentage imposed to the waist area and the stretching force in the stretched condition, with the use of illustration material, such as Microplast;

FIG. 7 is a graphic of the corrugation percentage imposed to the waist region and the stretching force in the relaxed condition with use of illustrative material, such as Microplast;

FIG. 8 is a graphic of the corrugation 10 percentage imposed to the waist region and the shearing force in the stretched condition, with use of illustrative material, such as Microplast;

FIG. 9 is a graphic of the corrugation percentage imposed to the waist region and the shearing force in the relaxed condition, with use of illustrative material, such as Microplast;

FIG. 10 is a graphic of the corrugation percentage imposed to the waist region and the shearing force in the relaxed condition, with use of explaining material, such as HTH 15, and

FIG. 11 is a graphic of the corrugation percentage imposed to the waist region and the stretching force in the relaxed condition, with use of explaining material, such as HTH 15.

### DETAILED DESCRIPTION OF THE INVENTION

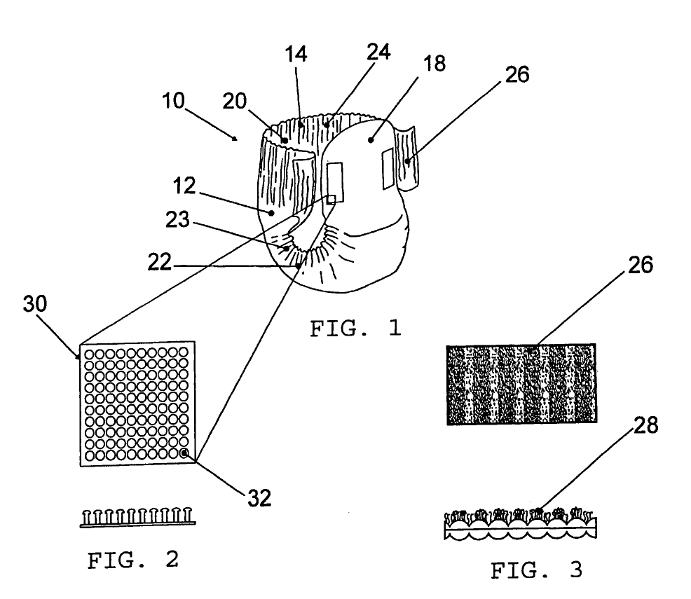

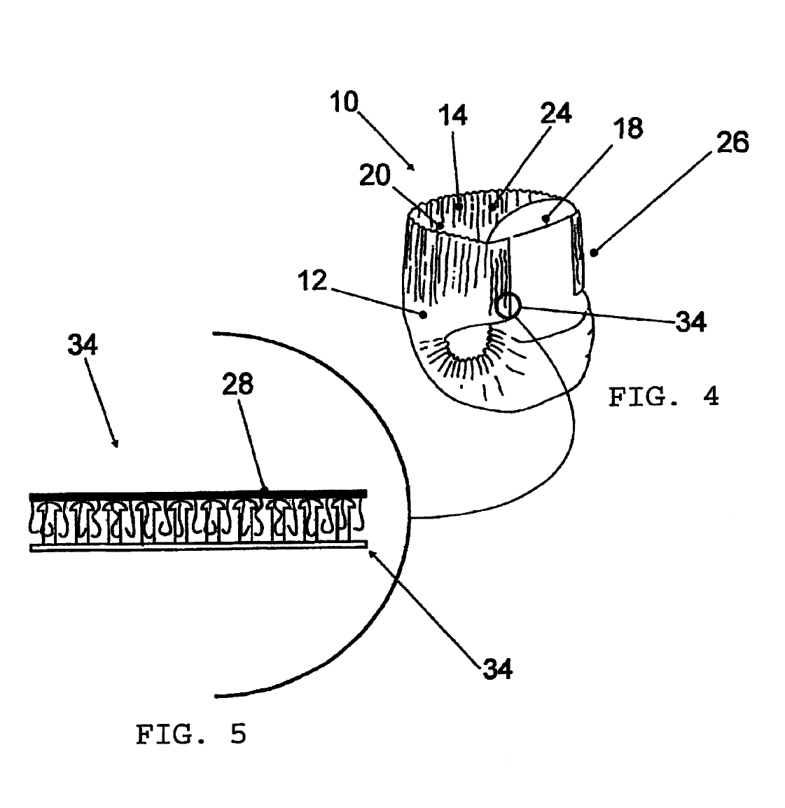

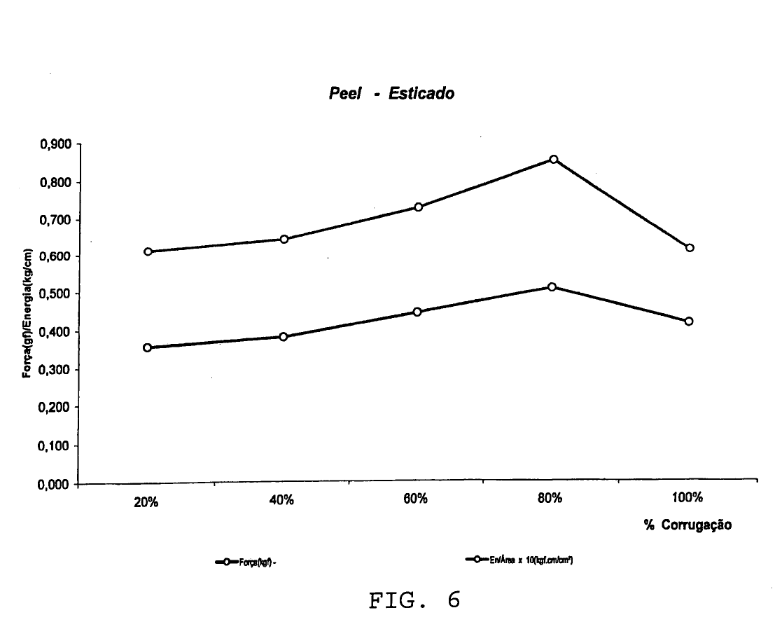

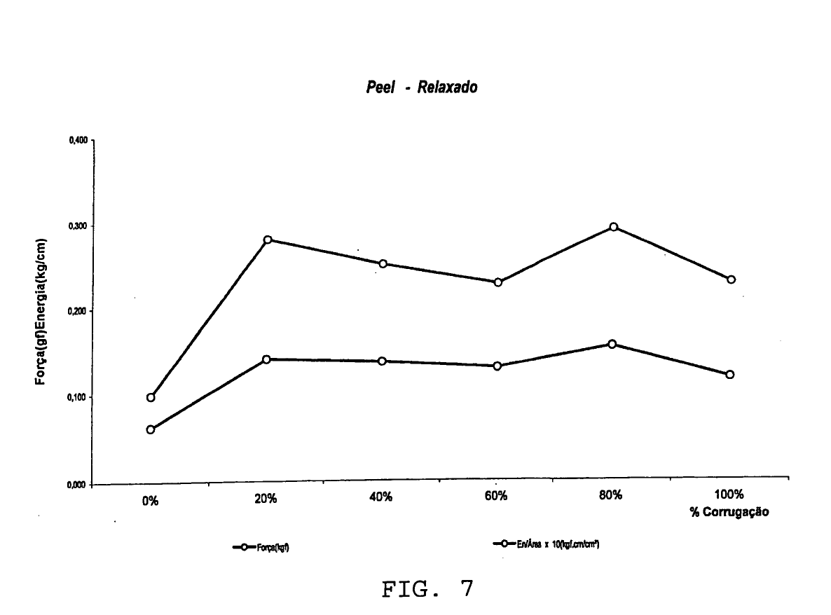

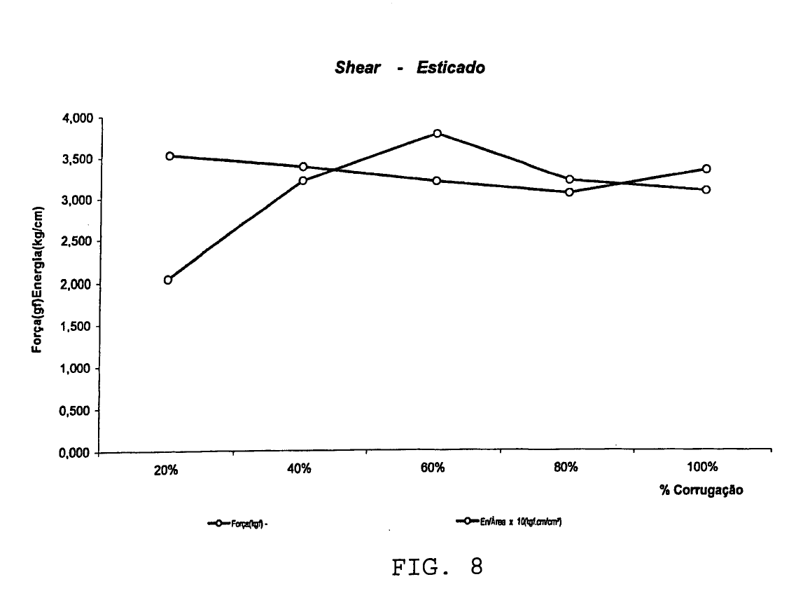

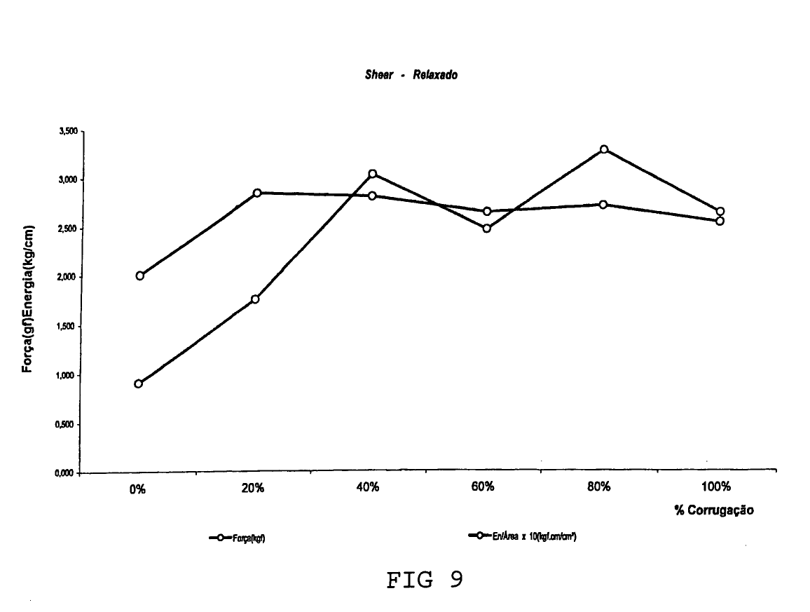

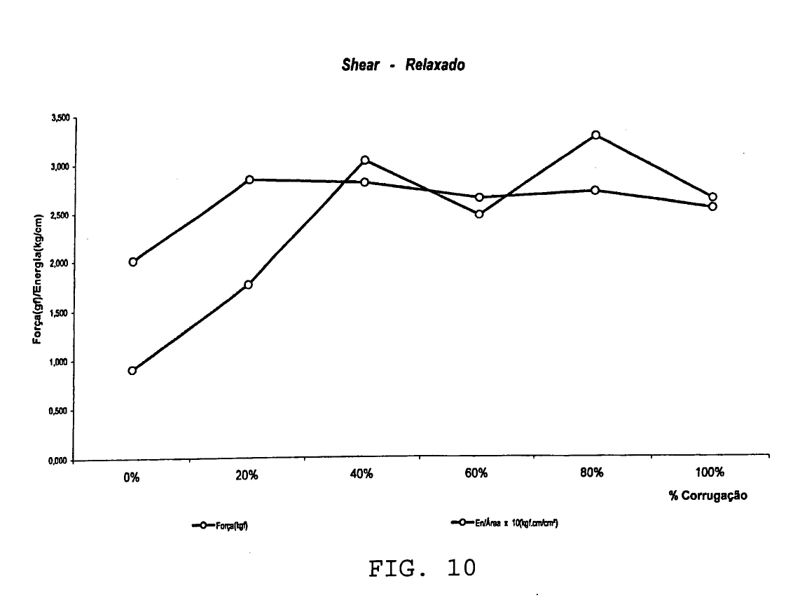

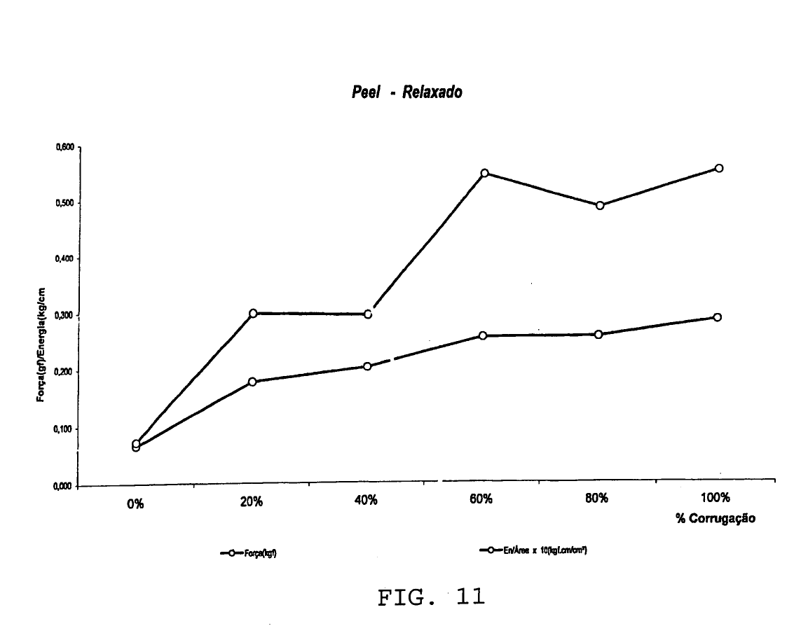

FIG. 1 of drawings illustrates a preferred modality of this invention, where an absorbing article can be seen, such as a diaper 10 with an external layer or sheet impermeable to liquid 12, an internal coating or sheet permeable to liquid 14 and a multi-layer absorbing core between the external coating 12 and the internal sheet 14, which may be internal not visible in the drawings.

Diaper 10 of this invention defines a frontal part 18, a rear part 20 and a part between the legs 22 between the front and rear parts. The part between the legs has elastics 23 for adjustments on the region of thighs of the user.

The external 12 and internal layers 14 of the absorbing article are tied with adhesive glue, thermal, sound or any other kind of adhesion known, such as, a continuous and uniform adhesive layer, a standard adhesive layer, a pulverized adhesive layer.

On the waist region 24 of the absorbing article, specifically on the rear part 20 of the absorbing article 10, between the referred to external layers 12 and internal 14, there are elastic components (not illustrated in the drawings), which are tensioned of stretched upon placement, in such a way that when the set of internal and external layers and elastic components is released, a spontaneous contraction occurs, forming a continues region of concentrated and corrugated fibers in the internal cover of sheet.

Sheet 14 can be corrugated, which provides a corrugated surface on the region of the waist 24 with more quantity of filaments or fibers in loop, adequate for fastening a component provided with multi-hooks. The level of corrugation depends on the stretching to which the elastic material is submitted, and it is possible to reach a corrugated surface with values between 1% and 100% of corrugation level.

The mechanical fastening system of this invention 10 is formed by a first corrugated superficial portion 26, continuous and disposed all over the surface of the waist region 24, formed by many filaments of loop elements 28 and by a second portion of fastening 30 formed by many hooks 32, the filaments of the loop elements 28 with a format adequate to gripping with the hooks 32.

In the preferential modality, the hook elements 32 have a generic form of a mushroom. Logically, such format is not restrictive, and it's possible the use of any suitable format that allows twisting and, consequently, the fastening of hooks into loops.

In this invention, the loop elements 28 are provided in the internal portion of the waste area 24, on the rear part 20 of the absorbing article 10, providing a continuous surface of fastening or dedicated region of fastening, which provides for a broad variation of fastening of the absorbing article on the user, without any limitations.

In the preferential modality of this invention, the hook elements 32 are foreseen in a fastening portion 30 located on the external portion of the frontal part 18 of the absorbing article 10, and may be disposed in a limited region by a wide extension of the surface dedicated to the fastening enabling the hook region to be restricted.

As mentioned above, in the preferential modality of this invention, the loop elements are formed in the permeable covering itself or blanket 14, and may be extended throughout the waist region of the absorbing article. Optionally, the loop elements may be foreseen in two or more dedicated regions in a reduced size, in opposite sides in the waist region of the absorbing article. Additionally, the dedicated regions of twisting filaments or loops, which may be manufactured with the same material as the sheet, the rest of the waist region may be manufactured out of another material such as a non-twisting plastic full the fastening of the absorbing article 10 with the fastening system of this invention is obtained after the placement of the referred article on the user' 5 body, by the juxtaposition of part of the corrugated surface 26, with loop elements 28 in the frontal part region 18 of the absorbing article containing a portion 30 with the hook elements 32, adjusting the size according to the user's waist size, 80 that the absorbing article is appropriately fixed to the user's body.

A person skilled in the technique will understand that the positioning of the loop elements in the internal part of the rear portion of the absorbing article and hook elements in the external part of the front part of the absorbing article may be exchanged depending on the manufacturer's convenience. Constructing variations of this kind are encompassed in the scope of this invention.

In the preferential modality, the loop elements are arranged in the internal part of the rear portion of the absorbing article (waist region) and the hook elements are anticipated for the external part of the front portion of the absorbing article, aiming at the practicality upon placement of the absorbing article on the user's body, by the juxtaposition of the rear to front hook, reaching mechanical stability as the junction point 34, closing point, is positioned in the frontal direction of the user's body the junction point 34 is the result of the intersection in the fastening of the portions of surfaces 30 and 26. This point 34 in relation to the user's body is presented in a non-variable manner on the sides of the frontal part 18. This occurs due to the presence in the region 30 in the lateral frontal part 18. Regardless of how well adjusted to the rear region 24 it may be (defining the variation of user's 5 waist perimeter) on the frontal part 18, the junction point 34 is not altered. Once the non-variable junction point 34, permanent on the sides of the frontal part 18 is obtained, there will be a better fastening of the absorbing device 10 to the user's body. This benefit is obtained due to the fact that the junction point 34 is permanently located on the sides of the frontal part 18, avoiding rotational dislocation of the absorbing article 10 on the user's body, originated by the weight of the urine absorbed by the absorbing core and by the contraction strength of the lateral elastics of the legs 23. This better fastening will avoid loosening of the fastening portions 26 and 30, avoiding leakage of urine and discomfort for users.

Although this invention has been described in details in relation to a preferential modality thereof, it must be understood by those skilled in the technique that following the understanding of the invention teachings it will be possible to reach modifications, alterations and equivalents of such modality. This way, the scope of this invention will be determined by the attached claims and their equivalents.

1. The mechanical fastening system for absorbing articles, with an external impermeable layer (12), a permeable internal layer (14) and an absorbing core sandwiched between them, the article defining the frontal (18) and rear (20) parts united by a part between the legs (22), the fastening system CHARACTERIZED in that it encompasses a first superficial portion (26) and a second fastening portion (30), the first superficial portion (26) located in the rear part (20) of the article, second fastening portion (30) placed on the sides of the frontal part (18) and formed by a plurality of projecting elements (32) fixable by juxtaposition of hooking elements (28).

2. The mechanical fastening system for absorbing articles, according to claim 1, CHARACTERIZED in that the plurality of the hooking elements are fibers or filaments in loop (28) formed in the internal surface of the permeable layer (14) by the corrugation obtained with the liberation of the stretching imposed to the elastic components sandwiched between the permeable (14) and impermeable (12) layers.

3. The mechanical fastening system for absorbing articles, according to claim 1, CHARACTERIZED in that the plurality of the projecting elements are elements in hook (32), placed in the hooking portion (30), placed in the external impermeable layer (12).

4. Mechanical fastening system for absorbing articles, according to claim 1, CHARACTERIZED in that the plurality of the loop filaments (28) is extended for at least one superficial region (26), and may reach its totality, forming a hooking region for the fastening of projecting elements (32) of the second portion (30).

5. Mechanical fastening system for absorbing articles, according to claim 1, CHARACTERIZED in that the plurality of the loop filament (28) is placed in the internal part of the free extremity of the rear part (20) of the absorbing article (10), the plurality of the projecting elements (32) is anticipated on the external surface of the frontal part (18), so that the plurality of filaments in loop (28) is juxtaposed over the plurality of the projecting elements (32) for the system fastening.
